(19) United States
(12) Patent Application Publication
(10) Pub. No.: US 2016/0249794 A1
(43) Pub. Date: Sep. 1, 2016

(54) ENDOSCOPE REPROCESSOR

(71) Applicant: OLYMPUS CORPORATION, Tokyo (JP)

(72) Inventor: Shintaro SUZUKI, Sagamihara-shi (JP)

(73) Assignee: OLYMPUS CORPORATION, Tokyo (JP)

(21) Appl. No.: 15/150,536

(22) Filed: May 10, 2016

Related U.S. Application Data
(63) Continuation of application No. PCT/JP2015/061633, filed on Apr. 15, 2015.

(30) Foreign Application Priority Data
Jun. 25, 2014 (JP) 2014-130625

(51) Int. Cl. A61B 1/12; C02F 1/00; B08B 3/08; B08B 3/10; B08B 9/023; B08B 9/032

(52) U.S. Cl. CPC A61B 1/123; A61B 1/125; B08B 9/023; B08B 9/0325; B08B 3/08; B08B 3/10; B08B 3/102; C02F 1/001; A61B 2090/701

(57) ABSTRACT

An endoscope reprocessor includes: a filtering tool including: a main body portion provided with a channel connecting a lead-in portion and a lead-out portion; a filter portion provided in the channel; and a first check valve provided in the channel; a processing tank; a tank that stores a disinfecting liquid; a lead-out conduit that guides the fluid led out from the lead-out portion to the processing tank; a second check valve with a distal end connected to the lead-out conduit; a first conduit with a distal end connected to the second check valve and a proximal end connected to the tank; and an injection pump that transfers the disinfecting liquid in the tank from the proximal end toward the distal end of the first conduit.

### CROSS REFERENCE TO RELATED APPLICATION

This application is a continuation application of PCT/JP2015/061633 filed on Apr. 15, 2015 and claims benefit of Japanese Application No. 2014-130625 filed in Japan on Jun. 25, 2014, the entire contents of which are incorporated herein by this reference.

### BACKGROUND OF THE INVENTION

1. Field of the Invention

The present invention relates to an endoscope reprocessor including a filter portion that filters a fluid.

2. Description of the Related Art

A cleaning process and a disinfecting process are applied to an endoscope used in a medical field after the endoscope is used. An endoscope cleaning/disinfecting apparatus that automatically executes the cleaning process and the disinfecting process of the endoscope is disclosed in, for example, Japanese Patent Application Laid-Open Publication No. 2013-135820.

The endoscope cleaning/disinfecting apparatus includes a filtering tool that filters water to be used. When the filtering tool is exchanged, a new filtering tool is attached, and then inside of a conduit of the endoscope cleaning/disinfecting apparatus where the filtered water passes is disinfected by using a disinfecting liquid stored in the apparatus. An endoscope cleaning/disinfecting apparatus 1 disclosed in FIG. 4 of Japanese Patent Application Laid-Open Publication No. 2013-135820 includes: a water filter 17; and a conduit 9 for leading water filtered by the water filter 17 into a cleaning tank 4.

### SUMMARY OF THE INVENTION

An aspect of the present invention provides an endoscope reprocessor including: a filtering tool including: a lead-in portion that leads in a fluid; a lead-out portion that leads out the fluid; a main body portion provided with a channel connecting the lead-in portion and the lead-out portion; a filter portion that is provided in the channel and filters the fluid flowing through the channel; and a first check valve that is provided between the filter portion and the lead-out portion in the channel and allows the fluid to pass only in a direction from the filter portion toward the lead-out portion; a processing tank that houses at least one of an endoscope and an endoscope accessory; a tank that stores a cleaning liquid or a disinfecting liquid; a lead-out conduit that is detachably connected to the lead-out portion of the filtering tool and guides the fluid led out from the lead-out portion to the processing tank; a second check valve with a distal end connected to the lead-out conduit, the second check valve allowing the fluid to pass only in a direction from a proximal end toward the distal end; a first conduit with a distal end connected to the second check valve and a proximal end connected to the tank; and an injection pump that transfers the cleaning liquid or the disinfecting liquid in the tank from the proximal end toward the distal end of the first conduit.

### BRIEF DESCRIPTION OF THE DRAWINGS

FIG. 1 is a diagram showing configurations of an endoscope reprocessor and a filtering tool for endoscope reprocessor of a first embodiment;

FIG. 2 is a diagram showing a detailed configuration of the filtering tool for endoscope reprocessor of the first embodiment;

FIG. 3 is a diagram showing channels in a water lead-in portion and the filtering tool for endoscope reprocessor of the first embodiment;

FIG. 4 is a diagram showing a modification of the first embodiment;

FIG. 5 is a diagram showing a configuration of the endoscope reprocessor of a second embodiment;

FIG. 6 is a diagram showing a configuration of the endoscope reprocessor of a third embodiment;

FIG. 7 is a diagram showing a configuration of the endoscope reprocessor of a fourth embodiment;

FIG. 8 is a diagram of a processing tank of the endoscope reprocessor of a fifth embodiment as viewed from above;

FIG. 9 is a diagram showing a configuration of the endoscope reprocessor of a sixth embodiment;

FIG. 10 is a diagram showing a configuration of a tray of the sixth embodiment;

FIG. 11 is a perspective view of the endoscope reprocessor of a seventh embodiment;

FIG. 12 is an enlarged view of a hinge of a front surface door of the seventh embodiment as viewed from above;

FIG. 13 is a diagram describing a water leak test by the endoscope reprocessor of an eighth embodiment; and

FIG. 14 is a partial enlarged view of a connection tube of the eighth embodiment.

### DETAILED DESCRIPTION OF THE PREFERRED EMBODIMENTS

Hereinafter, preferred embodiments of the present invention will be described with reference to the drawings. Note that, scaling of each constituent element varies in each drawing used in the following description in order to illustrate each constituent element in a size that allows recognizing the constituent element on the drawing, and the present invention is not limited only to quantities of the constituent elements, shapes of the constituent elements, ratios of the sizes of the constituent elements, and relative positional relationship between respective constituent elements described in the drawings.

### First Embodiment

Hereinafter, a first embodiment will be described as an example of the embodiments of the present invention. An endoscope reprocessor 1 is an apparatus that uses water, a cleaning liquid, a disinfecting liquid, and the like to apply one or a combination of a rinsing process, a cleaning process, and a disinfecting process to at least one of an endoscope and an endoscope accessory (the endoscope and the endoscope accessory are not shown).

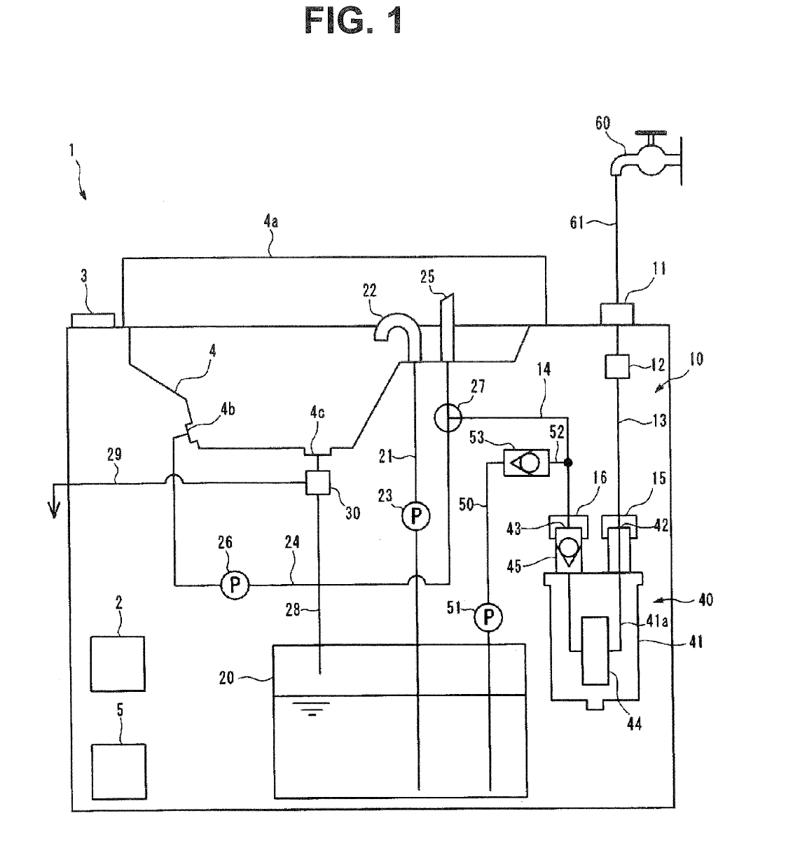

As shown in FIG. 1, the endoscope reprocessor 1 includes a control portion 2, an operation portion 3, a processing tank 4, a tank 20, and a water lead-in portion 10. A filtering tool for endoscope reprocessor 40 is detachably connected to the endoscope reprocessor 1.

The control portion 2 is an apparatus that controls action of each constituent element described later of the endoscope reprocessor 1 based on a predetermined program and is, for example, a computer including an arithmetic apparatus, a storage apparatus, an auxiliary storage apparatus, and an input and output apparatus.

The operation portion 3 includes one or a plurality of operation members, such as a lever switch, a dial switch, a button switch, and a touch sensor. The operation portion 3 is a part for a user to input an action instruction of the endoscope reprocessor 1.

The endoscope reprocessor 1 is provided with a power supply apparatus 5 that supplies electric power to the control portion 2 and other constituent elements of the endoscope reprocessor 1. The power supply apparatus 5 may be configured to obtain electric power from a commercial power supply to supply the electric power to each constituent element or may be configured to include a primary battery, a secondary battery, or a power generation apparatus to supply electric power to each component.

The processing tank 4 has a concave shape including an opening portion opening upward and can house at least one of the endoscope and the endoscope accessory inside. The processing tank 4 is configured to be able to store a liquid inside.

In an example of the present embodiment, the opening portion of the processing tank 4 opening upward is provided with a lid member 4a. The lid member 4a opens and closes the opening portion of the processing tank 4. Through the opening portion of the processing tank 4, at least one of the endoscope and the endoscope accessory is housed inside of the processing tank 4 from the outside of the endoscope reprocessor 1.

A circulation port 4b and a drainage port 4c are provided at the law part of the processing tank 4. A nozzle 22 and a circulation nozzle 25 are installed in the processing tank 4. Although details will be described later, the nozzle 22 is a part that discharges a cleaning liquid or a disinfecting liquid stored in the tank 20 into the processing tank 4.

The circulation nozzle 25 is communicated with the circulation port 4b through a circulation conduit 24. A three-way valve 27 is installed between the circulation nozzle 25 and the circulation conduit 24. A distal end of a lead-out conduit 14 of the water lead-in portion 10 described later is connected to the three-way valve 27.

The three-way valve 27 can switch a state in which the circulation conduit 24 and the circulation nozzle 25 are communicated and a state in which the lead-out conduit 14 and the circulation nozzle 25 are communicated.

A circulation pump 26 is provided on the circulation conduit 24. When the three-way valve 27 is put into the state in which the circulation conduit 24 and the circulation nozzle 25 are communicated, and the circulation pump 26 is operated, the liquid in the processing tank 4 is sucked out from the circulation port 4b and then returns into the processing tank 4 through the circulation conduit 24 and the circulation nozzle 25. That is, the operation of the circulation pump 26 circulates the liquid stored in the processing tank 4 through the circulation conduit 24.

The endoscope reprocessor 1 houses at least one of the endoscope and the endoscope accessory in the processing tank 4 and circulates one of the water, the cleaning liquid, and the disinfecting liquid to thereby apply one of the rinsing process, the cleaning process, and the disinfecting process to at least one of the endoscope and the endoscope accessory.

The drainage port 4c is a part that exhausts the liquid stored in the processing tank 4 to the outside of the processing tank 4. When a cleaning liquid that can be repeatedly utilized or a disinfecting liquid that can be repeatedly utilized is used, a recovery conduit 28 for connecting the processing tank 4 and the tank 20 can also be provided.

The drainage port 4c is connected to the recovery conduit 28 and a drainage conduit 29 through a switch valve 30. The switch valve 30 can switch between a state in which the drainage port 4c is opened to make a connection with one of the recovery conduit 28 and the drainage conduit 29 and a state in which the drainage port 4c is closed.

The recovery conduit 28 connects the switch valve 30 and the tank 20.

When the drainage port 4c is opened to connect the drainage port 4c and the recovery conduit 28 in the state in which the cleaning liquid or the disinfecting liquid is stored in the processing tank 4, the cleaning liquid or the disinfecting liquid in the processing tank 4 is recovered into the tank 20. However, the cleaning liquid or the disinfecting liquid used in the present invention is not limited to the liquid that can be repeatedly utilized, and the liquid may be discarded after the liquid is used once.

The drainage conduit 29 extends to the outside of the endoscope reprocessor 1. When the drainage port 4c is opened to connect the drainage port 4c and the drainage conduit 29, the liquid stored in the processing tank 4 is exhausted to the outside of the endoscope reprocessor 1.

The tank 20 stores a disinfecting liquid, such as peracetic acid. Note that the tank 20 may be removable from the endoscope reprocessor 1.

Other than the recovery conduit 28, a liquid lead-in conduit 21 and a first conduit 50 are connected to the tank 20.

The liquid lead-in conduit 21 is a conduit for connecting the tank 20 and the nozzle 22. A liquid lead-in pump 23 is provided on the liquid lead-in conduit 21. When the liquid lead-in pump 23 is operated, the cleaning liquid or the disinfecting liquid stored in the tank 20 is discharged from the nozzle 22 through the liquid lead-in conduit 21 and is led into the processing tank 4.

A distal end of the first conduit 50 is connected to a second check valve 53, and a proximal end is connected to the tank 20. A liquid injection pump 51 is provided on the first conduit 50. When the liquid injection pump 51 is operated, the cleaning liquid or the disinfecting liquid stored in the tank 20 flows into the second check valve 53. That is, the liquid injection pump 51 is operated to transfer the cleaning liquid or the disinfecting liquid from the proximal end toward the distal end of the first conduit.

A distal end of the second check valve 53 is connected to the lead-out conduit 14 described later, and a proximal end is connected to the distal end of the first conduit 50. Note that a conduit for connecting the second check valve 53 and the lead-out conduit 14 may be provided between the distal end of the second check valve 53 and the lead-out conduit 14. In an example of the present embodiment, the distal end of the second check valve 53 and the lead-out conduit 14 are connected by a second conduit 52.

The second check valve 53 allows a fluid to pass only in a direction from the proximal end toward the distal end. That is, the second check valve 53 allows the fluid to pass in a direction from the first conduit 50 toward the lead-out conduit 14, but does not allow the fluid to pass in a direction from the lead-out conduit 14 toward the first conduit 50.

When the liquid injection pump 51 is operated, the cleaning liquid or the disinfecting liquid stored in the tank 20 passes through the first conduit 50 and the second check valve 53 and is injected into the lead-out conduit 14. On the other hand, the fluid in the lead-out conduit 14 does not flow into the first conduit 50 and the tank 20 through the second check valve 53.

The water lead-in portion 10 is a part that leads water supplied from a water supply portion 60 into the endoscope reprocessor 1. The filtering tool for endoscope reprocessor (hereinafter, simply called “filtering tool”) 40 including a filter portion 44 that filters the water is connected to the water lead-in portion 10. The filtering tool 40 can be attached to and detached from the water lead-in portion 10. The water supply portion 60 is, for example, a water faucet that discharges tap water.

The water lead-in portion 10 includes a tap water connection portion 11, a water lead-in valve 12, a lead-in conduit 13, and the lead-out conduit 14. The tap water connection portion 11 is a part connected with the water supply portion 60. A distal end of a hose 61 in which a proximal end is connected to the water faucet can be connected to the tap water connection portion 11, for example.

A proximal end of the lead-in conduit 13 is connected to the tap water connection portion 11 through the water lead-in valve 12. The water lead-in valve 12 is opened and closed to switch whether to allow the water supplied from the water supply portion 60 to flow into the lead-in conduit 13.

A distal end of the lead-in conduit 13 is provided with a lead-in side connector portion 15 for detachably connecting a lead-in portion 42 of the filtering tool 40. The lead-in portion 42 can be connected to the lead-in side connector portion 15 to lead the water into the filtering tool 40 from the lead-in conduit 13.

A distal end of the lead-out conduit 14 is connected to the three-way valve 27. A proximal end of the lead-out conduit 14 is provided with a lead-out side connector portion 16 for detachably connecting the lead-out portion 43 of the filtering tool 40. The lead-out portion 43 can be connected to the lead-out side connector portion 16 to lead filtered water led out from the filtering tool 40 into the lead-out conduit 14.

Therefore, when the water lead-in valve 12 is opened by putting the three-way valve 27 into the state in which the lead-out conduit 14 and the circulation nozzle 25 are communicated, the water supplied from the water supply portion 60 is led into the processing tank 4 through the lead-in conduit 13, the filtering tool 40, the lead-out conduit 14, and the circulation nozzle 25. That is, the lead-out conduit 14 is a conduit for guiding the fluid led out from the lead-out portion 43 of the filtering tool 40 to the processing tank 4. In this way, the water supplied from the water supply portion 60 can be led into the processing tank 4 after the water is filtered by the filtering tool 40.

As described, the distal end of the second check valve 53 is connected to the lead-out conduit 14. The second check valve 53 is connected to the lead-out conduit 14 in a section between the distal end and the proximal end of the lead-out conduit 14.

The filtering tool 40 includes the lead-in portion 42, the lead-out portion 43, a channel 41a, the filter portion 44, and a first check valve 45. The filtering tool 40 is formed by integrating and assembling each of these components (lead-in portion 42, lead-out portion 43, channel 41a, filter portion 44, and first check valve 45), and each of these components is removed when the filtering tool 40 connected to the endoscope reprocessor 1 is removed. Each of these components is connected when the filtering tool 40 is connected to the endoscope reprocessor 1.

As described, the lead-in portion 42 and the lead-out portion 43 can be detachably connected to the lead-in side connector portion 15 and the lead-out side connector portion 16 of the water lead-in portion 10, respectively. The channel 41a for connecting the lead-in portion 42 and the lead-out portion 43 is provided in the filtering tool 40. The channel 41a is provided with the filter portion 44 for filtering the liquid passing through the channel 41a. Therefore, the fluid led into the filtering tool 40 from the lead-in portion 42 flows through the channel 41a and is filtered by the filter portion 44. The fluid is then led out from the lead-out portion 43. The filter portion 44 is a so-called sterilizing filter and can filter liquid.

The first check valve 45 is installed between the filter portion 44 of the channel 41a and the lead-out portion 43 or installed on the lead-out portion 43. The first check valve 45 allows the fluid to pass in a direction from the filter portion 44 toward the lead-out portion 43, but does not allow the fluid to pass in a direction from the lead-out portion 43 toward the filter portion 44.

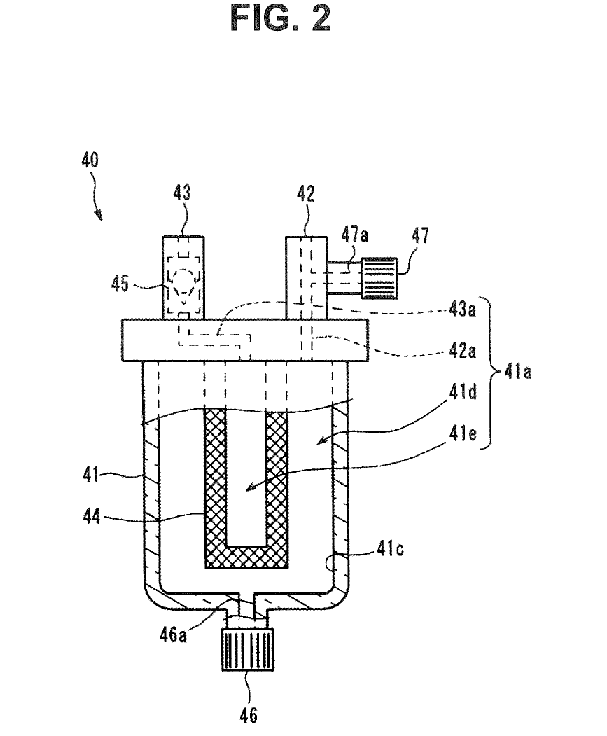

Hereinafter, a detailed configuration of the filtering tool 40 will be described. As shown in FIG. 2, the filtering tool 40 includes a container-like main body portion 41 provided with an internal space 41c for housing the filter portion 44 inside.

The internal space 41c is divided by the filter portion 44 into two spaces, a primary space 41d and a secondary space 41e. The fluid in the internal space 41c cannot move back and forth between the primary space 41d and the secondary space 41e without passing through the filter portion 44.

The filtering tool 40 is provided with a primary channel 42a, a secondary channel 43a, a drain channel 46a, and an air vent channel 47a for connecting an external space and the internal space 41c of the filtering tool 40. Note that the filtering tool 40 may not be provided with the drain channel 46a and the air vent channel 47a.

The primary channel 42a is a channel in which a proximal end opens into the lead-in portion 42, and a distal end opens into the primary space 41d of the internal space 41c. The secondary channel 43a is a channel separate and different from the primary channel 42a and is a channel in which a proximal end opens into the secondary space 41e of the internal space 41c, and a distal end opens into the lead-out portion 43.

That is, the channel 41a of the filtering tool 40 is formed by the primary channel 42a, the primary space 41d, the filter portion 44, the secondary space 41e, and the secondary channel 43a. The first check valve 45 is provided on the secondary channel 43a.

At least a section of the filtering tool 40 from the proximal ends of the filter portion 44, the secondary space 41e, and the secondary channel 43a to the first check valve 45 is sterilized.

The drain channel 46a is provided at a position that is a bottom portion of the main body portion 41 in a state that the filtering tool 40 is connected to the water lead-in portion 10, and the drain channel 46a connects the external space and the primary space 41d of the internal space 41c. The drain channel 46a is provided with a drain valve 46 for opening and closing the drain channel 46a.

The drain valve 46 is put into an open state only for exhausting the liquid in the internal space 41e to the outside and is put into a closed state in other cases. For example, when the filtering tool 40 is to be removed from the water lead-in portion 10, the drain valve 46 is put into the open state, because the liquid in the internal space 41c needs to be exhausted to the outside.

The air vent channel 47a is provided at a position that is an upper surface of the main body portion 41 in the state that the filtering tool 40 is connected to the water lead-in portion 10, and the air vent channel 47a connects the external space and the primary space 41d of the internal space 41c. The air vent channel 47a is provided with an air vent valve 47 for opening and closing the air vent channel 47a.

The air vent valve 47 is put into an open state only for exhausting the liquid in the internal space 41c to the outside and is put into a closed state in other cases. The air vent valve 47 can be put into the open state to quickly exhaust the liquid from the drain channel 46a, because the inside of the internal space 41c is opened to the atmosphere.

When the tank 20 stores the disinfecting liquid, a cleaning liquid tank for storing the cleaning liquid can also be provided separately from the tank 20. In this case, it is desirable to provide a conduit for connecting the cleaning liquid tank and the processing tank 4. When the cleaning liquid tank is arranged below the processing tank 4 in a gravity direction, it is desirable to provide a liquid sending pump on the cleaning liquid tank or the conduit.

When the tank 20 stores the cleaning liquid, a disinfecting liquid tank for storing the disinfecting liquid can also be provided separately from the tank 20. In this case, it is desirable to provide a conduit for connecting the disinfecting liquid tank and the processing tank 4. When the disinfecting liquid tank is arranged below the processing tank 4 in the gravity direction, it is desirable to provide a liquid sending pump on the disinfecting liquid tank or the conduit.

Note that although the endoscope reprocessor 1 can also include a component for sending the air into the processing tank 4 and other components in addition to the components described above, the components are the same as in an already-known endoscope reprocessor and will not be described.

Action in exchanging the filtering tool 40 of the endoscope reprocessor 1 of the present embodiment with the configuration will be described.

To exchange the filtering tool 40, the user first puts the air vent valve 47 and the drain valve 46 into the open state to remove the water from the inside of the water lead-in portion 10 and the inside of the used filtering tool 40 mounted on the water lead-in portion 10.

Next, the user removes the used filtering tool 40 from the water lead-in portion 10. Then, the user mounts a new filtering tool 40 on the water lead-in portion 10.

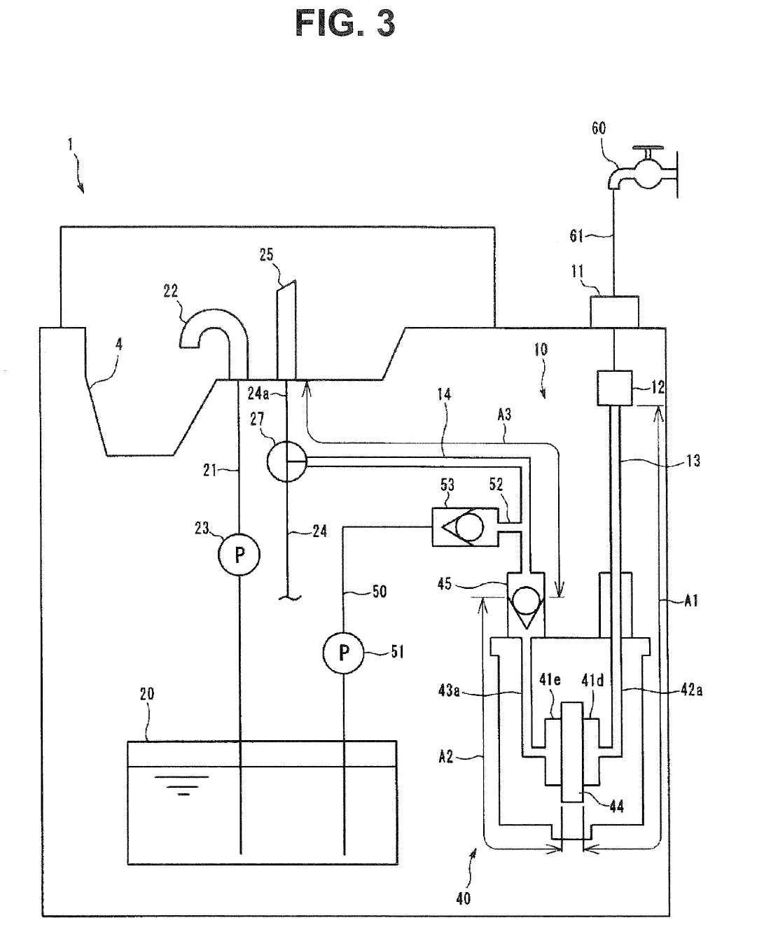

For the description, a plurality of regions A1, A2, A3, and A4 are set as shown in FIG. 3, for regions in which the water in the water lead-in portion 10 and the filtering tool 40 flows.

The region A1 includes the lead-in conduit 13, the primary channel 42a, and the primary space 41d. Air including germs and dirt enters the region A1 by removing the filtering tool 40 from the water lead-in portion 10. However, the region A1 is a part where the water before filtering by the filter portion 44 comes in contact at the execution of the rinsing process, the cleaning process, and the disinfecting process by the endoscope reprocessor 1, and the disinfecting process does not have to be executed after the region A1 comes into contact with the air including the germs and dirt.

The region A2 includes the secondary space 41e and a section from the proximal end of the secondary channel 43a to the first check valve 45. That is, the region A2 is a section between the filter portion 44 and the first check valve 45 in the channel 41a of the filtering tool 40.

In the region A2, the fluid does enter from the side provided with the first check valve 45. That is, only the fluid filtered by the filter portion 44 enters into the region A2. Furthermore, the region A2 is already in a sterilized state. Therefore, the region A2 is always kept in the sterilized state both in the state in which the filtering tool 40 is removed from the water lead-in portion 10 and the state in which the filtering tool 40 is mounted on the water lead-in portion 10. As a result, the disinfecting process does not have to be executed in the region A2 in exchanging the filtering tool 40. That is, the disinfecting liquid does not have to be sent into the region A2 after the exchange of the filtering tool 40.

The region A3 includes: a section of the secondary channel 43a from the first check valve 45 to the distal end; the lead-out conduit 14; the second conduit 52 that is a space from the lead-out conduit 14 to the second check valve 53; and the conduit 24a connecting the three-way valve 27 and the circulation nozzle 25. Germs and dirt in the outside air enter the region A3 by removing the filtering tool 40 from the water lead-in portion 10.

The region A3 is a part where the water filtered by the filter portion 44 comes in contact at the execution of the rinsing process, the cleaning process, or the disinfecting process by the endoscope reprocessor 1, and the cleaning process or the disinfecting process needs to be executed after the region A3 comes in contact with the air including germs and dirt.

Therefore, after the new filtering tool 40 is mounted on the water lead-in portion 10, the cleaning process or the disinfecting process of the region A3 of the water lead-in portion 10 is executed before the execution of the rinsing process, the cleaning process, or the disinfecting process by the endoscope reprocessor 1. For example, the user operates the operation portion 3 and inputs an instruction for executing the cleaning process or the disinfecting process of the water lead-in portion 10 to thereby execute a cleaning processing step or a disinfecting processing step of the region A3 described below.

Note that the endoscope reprocessor 1 may include a sensor that senses removal of the filtering tool 40 from the water lead-in portion 10, and the cleaning processing step or the disinfecting processing step of the region A3 may be automatically executed after the detection of the removal of the filtering tool 40. The endoscope reprocessor 1 may include a sensor that senses removal of the filtering tool 40 from the water lead-in portion 10, and the execution of the rinsing process, the cleaning process, or the disinfecting process may be prohibited after the detection of the removal of the filtering tool 40, until the end of the execution of the cleaning processing step or the disinfecting processing step of the region A3 based on the instruction by the user.

In the cleaning process or the disinfecting process of the region A3, the three-way valve 27 is put into the state in which the lead-out conduit 14 and the circulation nozzle 25 are communicated, and the liquid injection pump 51 is driven. When the liquid injection pump 51 is driven, the cleaning liquid or the disinfecting liquid stored in the tank 20 is led into the region A3 through the first conduit 50 and the second check valve 53. The liquid injection pump 51 is driven only for a predetermined time period until the region A3 is filled with the cleaning liquid or the disinfecting liquid. A state in which the region A3 is filled with the disinfecting liquid is kept for a predetermined time period, and the cleaning process or the disinfecting process in the region A3 is completed.

Whether to apply the cleaning process or to apply the disinfecting process to the region A3 can be appropriately selected according to a purpose. For example, it is desirable to apply the disinfecting process to the region A3 to disinfect the endoscope. It is desirable to apply the cleaning process or the disinfecting process to the region A3 to clean the endoscope.

After the cleaning process or the disinfecting process in the region A3 is finished, the water lead-in valve 12 is put into the open state for a predetermined time period to pour the water into the water lead-in portion 10 and the filtering tool 40 to flush out the cleaning liquid or the disinfecting liquid in the region A3. As a result of the action, the exchange of the filtering tool 40 in the endoscope reprocessor 1 is finished.

As described, the filter portion 44 is housed in the container-like main body portion 41 in the filtering tool 40 of the present embodiment, the first check valve 45 is provided in the section of the channel 41a from the filter portion 44 to the lead-out portion 43, and the first check valve 45 blocks the inflow of the fluid into the main body portion 41 from the lead-out portion 43. As a result, the region A2 in the filtering tool 40 is always kept in the sterilized state, and the inside of the filtering tool 40 does not have to be cleaned or disinfected before the start of the use of the new filtering tool 40. Therefore, in the filtering tool 40 of the present embodiment, the entire channel 41a of the filtering tool 40 does not have to be filled with the cleaning liquid or the disinfecting liquid in exchanging the filtering tool 40, and a time period required for exchange operation of the filtering tool 40 can be reduced. Since the entire channel 41a of the filtering tool 40 does not have to be filled with the cleaning liquid or the disinfecting liquid in exchanging the filtering tool 40, a used amount of the cleaning liquid or the disinfecting liquid used after the exchange of the filtering tool 40 can be reduced.

The endoscope reprocessor 1 of the present embodiment further includes the first conduit 50 and the disinfecting liquid injection pump 51 that directly send the cleaning liquid or the disinfecting liquid from the tank 20 into the lead-out conduit 14 connected to the lead-out portion 43 of the filtering tool 40. When the cleaning process or the disinfecting process is to be executed after the exchange of the filtering tool 40, it is only necessary that only the region A3 mainly including the lead-out conduit 14 is filled with the disinfecting liquid. Therefore, an amount of the cleaning liquid or the disinfecting liquid used in exchanging the filtering tool 40 can be reduced in the endoscope reprocessor 1 of the present embodiment.

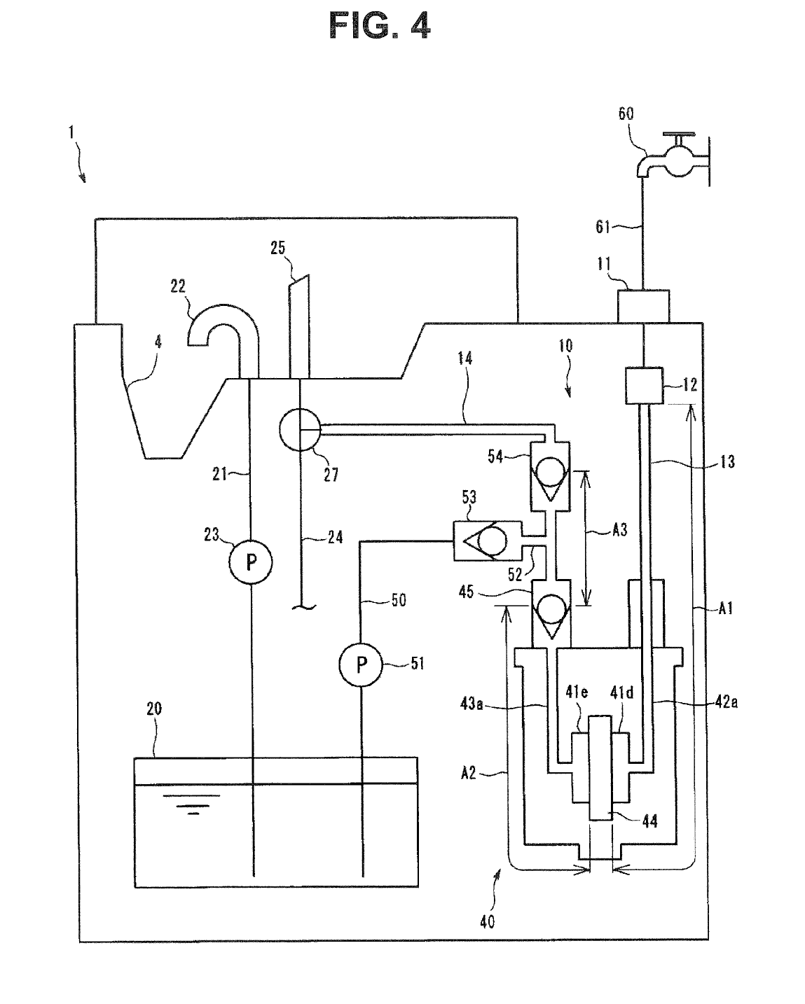

Next, FIG. 4 shows a modification of the endoscope reprocessor 1 of the present embodiment. The modification shown in FIG. 4 is different from the embodiment described above in that the lead-out conduit 14 is provided with a third check valve 54.

The third check valve 54 is provided in a section of the lead-out conduit 14, from a part where the distal end of the second check valve 53 is connected, to a distal end connected to the three-way valve 27. The third check valve 54 allows the fluid to pass in a direction from the proximal end toward the distal end in the lead-out conduit 14, but does not allow the fluid to pass in a direction from the distal end toward the proximal end. That is, the third check valve 54 allows the fluid to pass only in a direction from the first check valve 45 toward the three-way valve 27 in the lead-out conduit 14.

In the present modification, the region A3 includes a section from the first check valve 45 of the secondary channel 43a to the distal end, a section from the proximal end of the lead-out conduit 14 to the check valve 54, and the second conduit 52 that is a space from the lead-out conduit 14 to the second check valve 53.

Therefore, in the present modification, the third check valve 54 is provided on the lead-out conduit 14, and a volume inside of the region A3 that comes into contact with the air including germs and dirt in exchanging the filtering tool 40 can be smaller than in the embodiment described above. As a result, according to the endoscope reprocessor 1 of the present modification, the time period for filling the region A3 with the cleaning liquid or the disinfecting liquid can be further reduced in exchanging the filtering tool 40, and the time period required for the exchange operation of the filtering tool 40 can be further reduced. Furthermore, the amount of cleaning liquid or disinfecting liquid used after the exchange of the filtering tool 40 can be further reduced.

### Second Embodiment

Next, a second embodiment of the present invention will be described. Only differences from the first embodiment will be described below. The same constituent elements as in the first embodiment are designated with the same reference signs, and the description will be appropriately omitted.

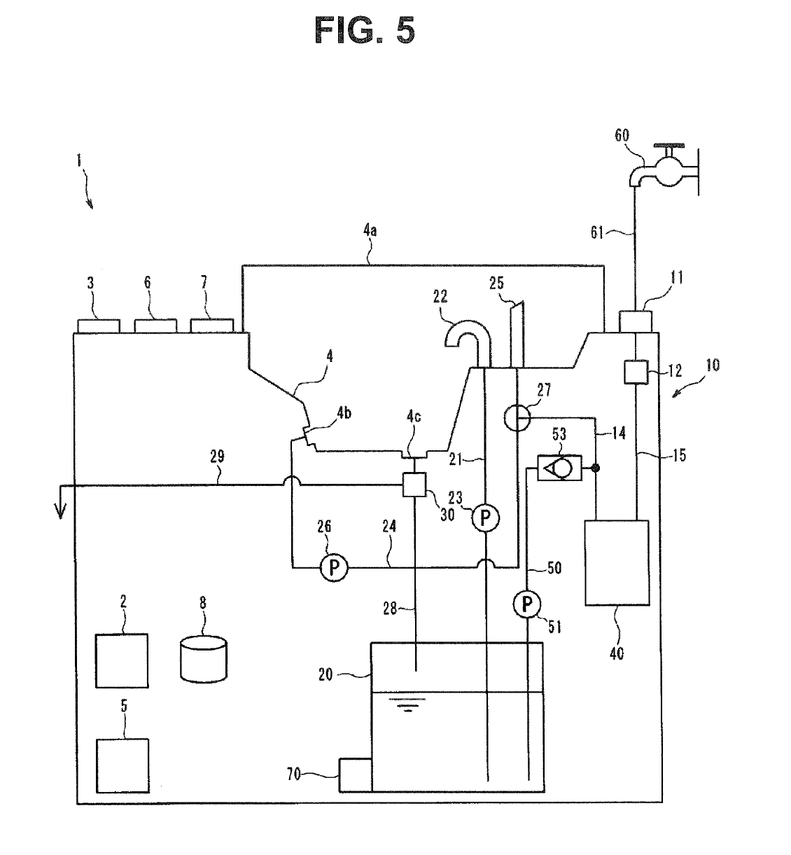

As shown in FIG. 5, the endoscope reprocessor 1 of the present embodiment includes a concentration measurement portion 70 that measures a concentration of the cleaning liquid or the disinfecting liquid. The concentration measurement portion 70 is provided on, for example, the tank 20 and can measure the concentration of the cleaning liquid or the disinfecting liquid in the tank 20.

The endoscope reprocessor 1 further includes an output portion 6, an ID input portion 7, and a storage portion 8. The output portion 6 includes an apparatus, such as a light emitting apparatus and an image display apparatus, that outputs visual information and an apparatus that outputs auditory information, such as warning sound and voice. The output portion 6 includes an image display apparatus in an example of the present embodiment.

The ID input portion 7 inputs ID information associated with an endoscope, a user, a subject for which the endoscope is used, and the like. The ID input portion 7 is, for example, a keyboard, and the user may manually input the ID information. The ID information may be recorded in, for example, a magnetic card, a bar code, a two-dimensional code, an RFID tag, or the like, and the ID input portion 7 may be configured to read the ID information. The ID input portion 7 is an RFID reader in an example of the present embodiment. The storage portion 8 is, for example, a hard disk drive or a flash memory.

In the present embodiment, before the execution of the concentration measurement step of the disinfecting liquid using the concentration measurement portion 70, the ID information of the user that carries out the concentration measurement step is inputted from the ID input portion 7. After the ID information of the user is inputted by the ID input portion 7, the concentration measurement step of the cleaning liquid or the disinfecting liquid using the concentration measurement portion 70 is executed. The control portion 2 stores a result of the concentration measurement step, that is, a numerical value of the concentration of the cleaning liquid or the disinfecting liquid, in the storage portion 8 in association with the ID information of the user that has carried out the concentration measurement step. The control portion 2 also stores date and time of the execution of the concentration measurement step in the storage portion 8.

The control portion 2 then determines whether the numerical value of the concentration of the cleaning liquid or the disinfecting liquid obtained in the concentration measurement step is a value that allows using the cleaning liquid or the disinfecting liquid.

If the concentration of the cleaning liquid or the disinfecting liquid is a value that does not allow the use, the control portion 2 outputs a warning from the output portion 6. For example, the output portion 6 that is an image display apparatus displays a character string and an image for visually indicating a reduction in the concentration of the cleaning liquid or the disinfecting liquid. In this case, a warning for prompting the exchange of the cleaning liquid or the disinfecting liquid may be displayed on the output portion 6 at the same time. The control portion 2 prohibits the execution of the rinsing process, the cleaning process, or the disinfecting process for at least one of the endoscope and the endoscope accessory and the execution of the cleaning process or the disinfecting process of the region A3. The prohibition state is continued until the concentration measurement step is executed again and it is judged that the concentration of the cleaning liquid or the disinfecting liquid is a value that allows the use.

In the present embodiment, the control portion 2 judges whether an elapsed time from execution of a latest concentration measurement step is equal to or greater than a predetermined time period, before the execution of the rinsing process, the cleaning process, and the disinfecting process for at least one of the endoscope and the endoscope accessory and before the execution of the disinfecting process of the region A3.

If the elapsed time from the execution of the latest concentration measurement step is equal to or greater than the predetermined time period, there is a possibility that the disinfecting liquid is degraded over time. Therefore, the control portion 2 prohibits the execution of the rinsing process, the cleaning process, and the disinfecting process for at least one of the endoscope and the endoscope accessory and the execution of the cleaning process or the disinfecting process of the region A3. At this point, the control portion 2 outputs a warning for re-execution of the concentration measurement step from the output portion 6.

If the elapsed time from the execution of the latest concentration measurement step is shorter than the predetermined time period, and the concentration of the disinfecting liquid is a concentration that allows the use, the execution of the rinsing process, the cleaning process, or the disinfecting process for at least one of the endoscope and the endoscope accessory and the execution of the disinfecting process of the region A3 are possible based on an instruction by the user. In this case, the concentration measurement step can also be associated with the cleaning process or the disinfecting process. The concentration measurement step may also be executed after the execution of the cleaning process or the disinfecting process, and if the concentration of the cleaning liquid or the disinfecting liquid indicates a result that allows the use, the association with the cleaning process or the disinfecting process is also possible in the same way. If the concentration of the cleaning liquid or the disinfecting liquid indicates a result that does not allow the use, a warning for prompting the exchange of the cleaning liquid or the disinfecting liquid is outputted, and a warning for re-execution of the cleaning process or the disinfecting process is also outputted again at the same time.

The ID input portion 7 of the endoscope reprocessor 1 of the present embodiment also reads the ID information provided to the endoscope subject to the rinsing process, the cleaning process, or the disinfecting process, the ID information provided to the subject for which the endoscope is used, and the ID information provided to the user that has confirmed normal execution of the rising process, the cleaning process, or the disinfecting process. The endoscope reprocessor 1 associates the ID information and stores the ID information in the storage portion 8. Note that the endoscope reprocessor 1 may include a lock mechanism that fixes the closed state of the lid member 4a, and if the ID information is not read by the ID input portion 7, the lock mechanism may maintain the fixed state of the closed state of the lid member 4a. This can prevent neglect of input of the ID information.

### Third Embodiment

Next, a third embodiment of the present invention will be described. Only differences from the first embodiment will be described below. The same constituent elements as in the first embodiment are designated with the same reference signs, and the description will be appropriately omitted.

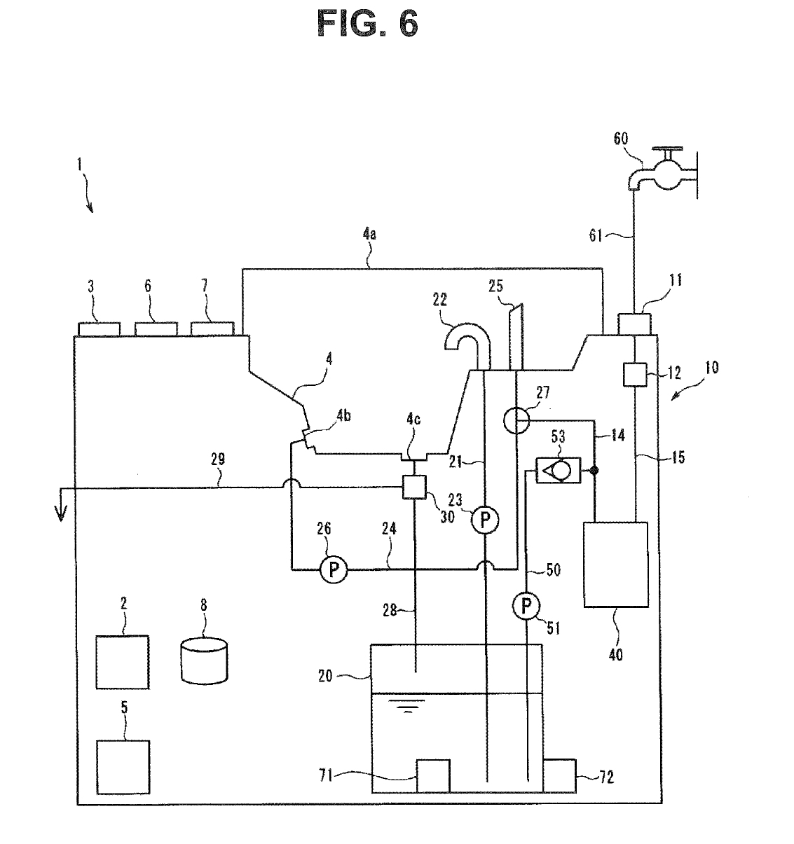

As shown in FIG. 6, the endoscope reprocessor 1 of the present embodiment includes a heater 71 and a temperature measurement portion 72. The heater 71 is an apparatus that heats the disinfecting liquid stored in the tank 20. The temperature measurement portion 72 is an apparatus that measures a temperature of the cleaning liquid or the disinfecting liquid stored in the tank 20.

In the present embodiment, the control portion 2 measures the temperature of the cleaning liquid or the disinfecting liquid by the temperature measurement portion 72 after the execution of the cleaning process or the disinfecting process for at least one of the endoscope and the endoscope accessory. If the temperature of the cleaning liquid or the disinfecting liquid is lower than a predetermined temperature, the control portion 2 operates the heater 71 to heat the cleaning liquid or the disinfecting liquid stored in the cleaning liquid or disinfecting liquid tank 20. Note that it is desirable that the heating by the heater 71 is performed such that the temperature of the cleaning liquid or the disinfecting liquid does not reach a temperature that degrades the cleaning liquid or the disinfecting liquid.

In this way, the heater 71 heats the cleaning liquid or the disinfecting liquid stored in the disinfecting liquid tank 20 after the execution of the cleaning process or the disinfecting process, and a waiting time for heating the cleaning liquid or the disinfecting liquid to a proper temperature in the execution of the next cleaning process or disinfecting process can be reduced, particularly when the temperature is low.

Note that the heater 71 stops the operation if the next cleaning process or disinfecting process is not executed even after a predetermined time period from the start of the operation. That is, if the waiting time period of the endoscope reprocessor 1 exceeds a predetermined time period, the heater 71 is stopped to reduce power consumption.

### Fourth Embodiment

Next, a fourth embodiment of the present invention will be described. Only differences from the first embodiment will be described below. The same constituent elements as in the first embodiment are designated with the same reference signs, and the description will be appropriately omitted.

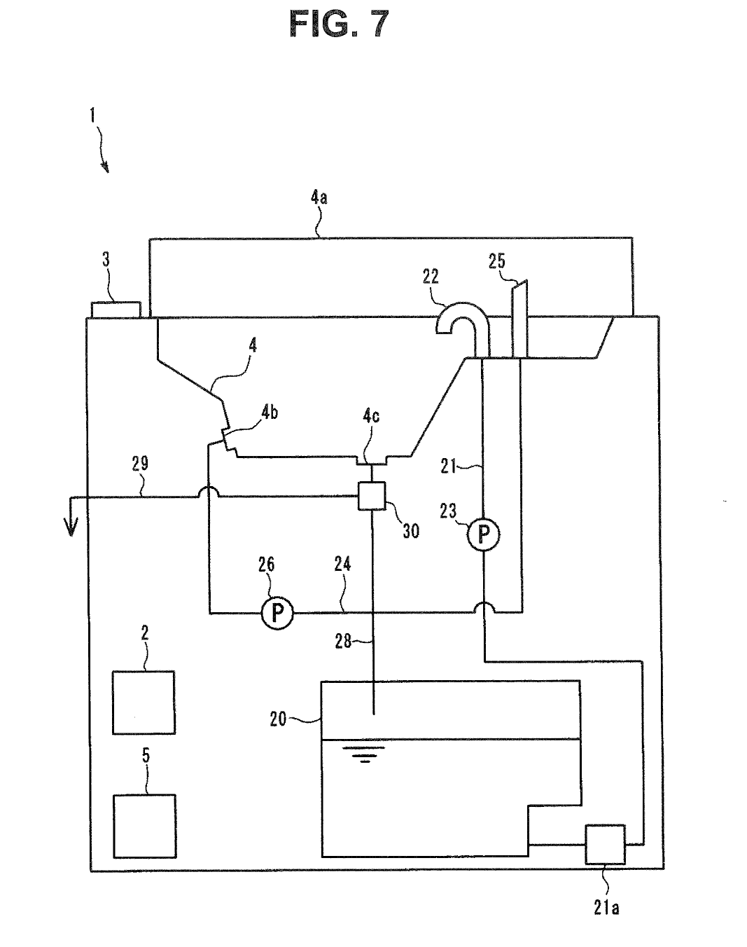

As shown in FIG. 7, the liquid lead-in conduit 21 is connected to a bottom surface portion of the tank 20 in the endoscope reprocessor 1 of the present embodiment. Therefore, an outer circumferential face of the liquid lead-in conduit 21 does not come in contact with the cleaning liquid or the disinfecting liquid. As a result, the outer circumferential face of the liquid lead-in conduit 21 is not degraded by the contact with the cleaning liquid or the disinfecting liquid, and periodical exchange of the liquid lead-in conduit 21 is not necessary.

A filter 21a for filtering the cleaning liquid or the disinfecting liquid is further installed on the liquid lead-in conduit 21. The filter 21a is arranged outside of the tank 20, and exchange operation when there is clogging or the like can be easily performed.

### Fifth Embodiment

Next, a fifth embodiment of the present invention will be described. Only differences from the first embodiment will be described below. The same constituent elements as in the first embodiment are designated with the same reference signs, and the description will be appropriately omitted.

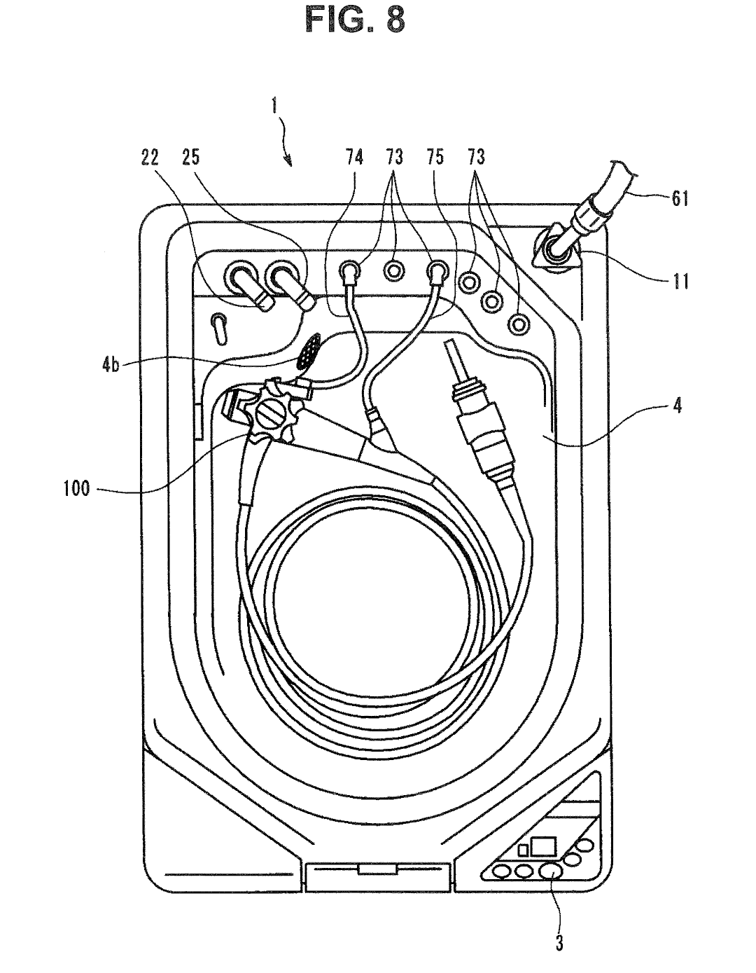

FIG. 8 is a diagram of the processing tank 4 of the endoscope reprocessor 1 of the present embodiment as viewed from above. A plurality of connector portions 73 for sending the liquid into the conduits of an endoscope 100 is installed in the processing tank 4.

Proximal end portions of connection tubes 74 and 75 can be connected to the connector portions 73. Distal end portions of the connection tubes 74 and 75 are connected to pipe sleeve portions of different conduits provided on the endoscope 100, respectively. For example, the distal end portion of the connection tube 74 is connected to a pipe sleeve portion of an air/water feeding conduit of the endoscope 100, and the distal end portion of the connection tube 75 is connected to a pipe sleeve portion of a treatment instrument insertion conduit of the endoscope 100.

The plurality of connector portions 73 are connected to conduits and pumps not shown, and the plurality of connector portions 73 discharge the liquid sucked out from the processing tank 4 through the circulation port 4b. The endoscope reprocessor 1 includes a flow rate measurement portion that measures a flow rate of the liquid discharged from each of the plurality of connector portions 73. The control portion 2 judges whether the connection tubes 74 and 75 are correctly connected to the connector portions 73 and the pipe sleeve portions of the endoscope 100 based on values of the flow rate measured by the flow rate measurement portion.

For example, if the distal end portion of the connection tube 74 is out of the pipe sleeve portion of the endoscope 100, a resistance is lower than when the distal end portion is normally connected, and the flow rate of the liquid discharged from the connector portion 73 increases. In this way, if the flow rate of the liquid discharged from the connector portion 73 is higher than a threshold that is a predetermined value, the control portion 2 judges that the distal end portions of the connection tubes 74 and 75 are not correctly connected to the pipe sleeve portions. However, the rates of the flow into the connected conduits vary in the conventional techniques, and the threshold of the flow rate used to judge whether the connection is correct needs to be set for each conduit. Furthermore, the flow rate is too low when the conduit is thin, and the judgement cannot be performed in some cases.

In the present embodiment, holes are provided on the cleaning tubes 74 and 75, and there is always a flow rate greater than a certain flow rate when the cleaning tubes 74 and 75 are connected to the connector portions 73. The threshold of the flow rate used to judge whether the cleaning tubes 74 and 75 are correctly connected to the connector portions 73 is set to the certain flow rate value.

That is, the flow rate of the liquid flowing in each of the connection tubes 74 and 75 is greater than the certain value, no matter to which one of the plurality of connector portions 73 the proximal end portion of the connection tube 74 in which the distal end portion is connected to the air/water feeding conduit and the proximal end portion of the connection tube 75 in which the distal end portion is connected to the treatment instrument insertion conduit are connected.

Therefore, according to the present embodiment, the control portion 2 can judge whether the connection tubes 74 and 75 are correctly connected to the connector portions 73 and the pipe sleeve portions of the endoscope 100 based on the values of the flow rate measured by the flow rate measurement portion, no matter to which one of the plurality of connector portions 73 the proximal end portions of the connection tubes 74 and 75 connected to different conduits of the endoscope 100 are connected.

### Sixth Embodiment

Next, a sixth embodiment of the present invention will be described. Only differences from the first embodiment will be described below. The same constituent elements as in the first embodiment are designated with the same reference signs, and the description will be appropriately omitted.

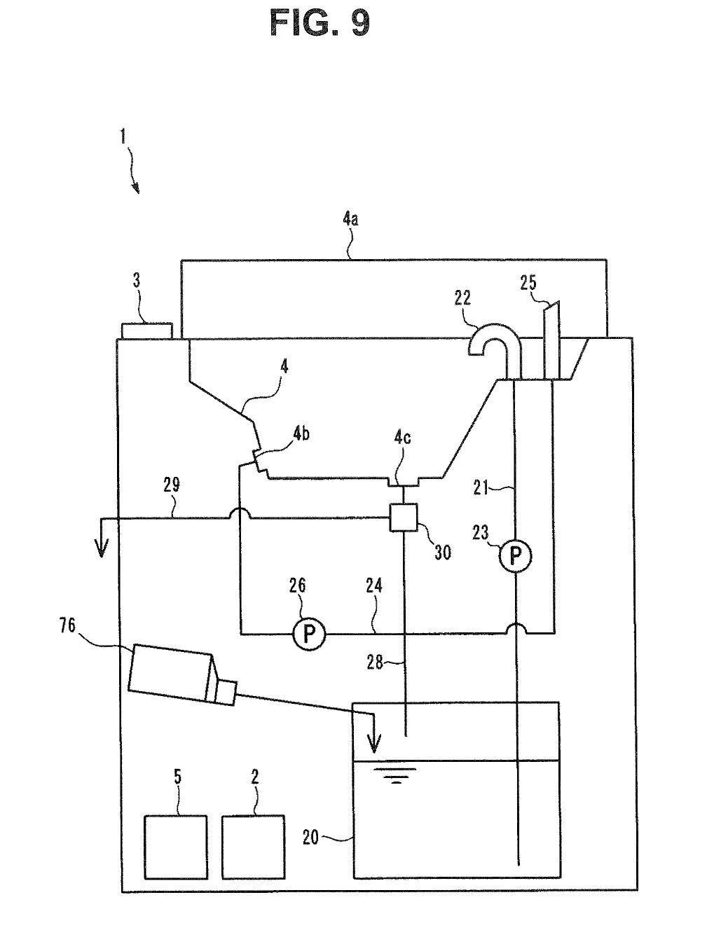

As shown in FIG. 9, the endoscope reprocessor 1 of the present embodiment is configured to supply the cleaning liquid or the disinfecting liquid into the tank 20 from one or a plurality of bottles 76 that can be attached to and detached from an apparatus main body. The cleaning liquid or the disinfecting liquid in the bottle 76 flows down into the tank 20 by gravity through a liquid supply conduit 77.

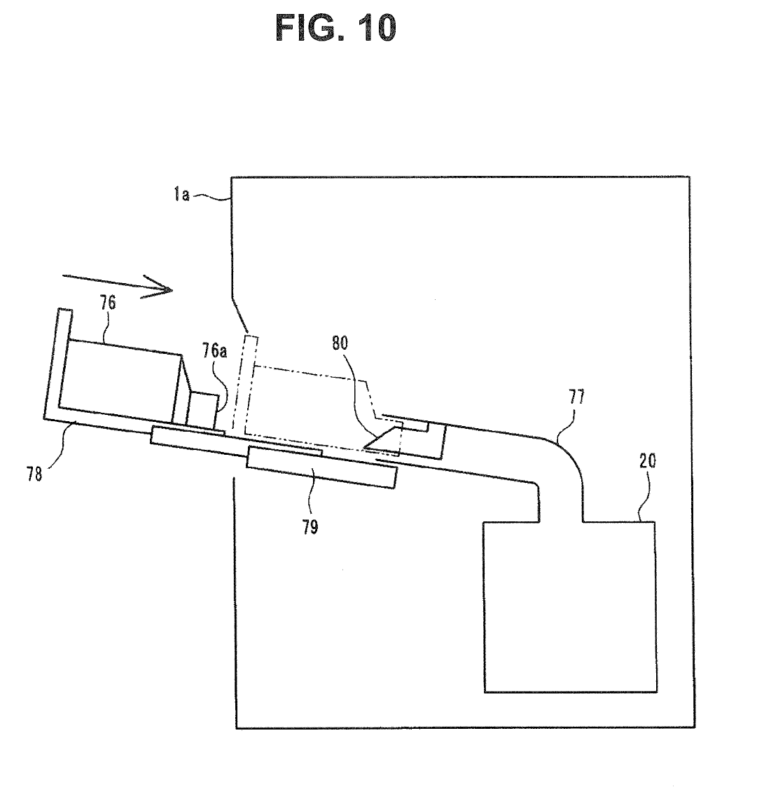

As shown in FIG. 10, the bottle 76 is placed on a tray 78 that can be drawn out from a side surface of an apparatus main body 1a. The tray 78 substantially linearly moves along a slide rail 79, between a position for storing the tray 78 in the apparatus main body 1a and a position protruding from the side surface of the apparatus main body 1a.

When the tray 78 is drawn out from the apparatus main body 1a, the tray 78 protrudes from the side surface of the apparatus main body 1a at an angle diagonally above a horizontal plane. In other words, when the tray 78 is to be pushed, from the state in which the tray 78 is drawn out to the state in which the tray 78 is stored in the apparatus main body 1a, the tray 78 moves diagonally downward as the tray 78 is pushed.

To transfer the cleaning liquid or the disinfecting liquid from the bottle 76 to the tank 20, the tray 78 is drawn out from the apparatus main body 1a, and the bottle 76 is mounted on the tray 78. Here, the bottle 76 is mounted in a posture such that a lid portion 76a faces a travelling direction in pushing the tray 78 into the apparatus main body 1a.

A penetration portion 80 piercing through the lid portion 76a of the bottle 76 when the tray 78 provided with the bottle 76 is pushed is fixed in the apparatus main body 1a. Therefore, when the tray 78 mounted with the bottle 76 is pushed into the apparatus main body 1a, the penetration portion 80 unseals the bottle 76. The cleaning liquid or the disinfecting liquid flown out from the unsealed bottle 76 flows down into the tank 20 through the liquid supply conduit 77.

In the endoscope reprocessor 1 of the present embodiment, the tray 78 is linearly pushed diagonally downward, and the penetration portion 80 can be stuck into the lid portion 76a of the bottle 76 with smooth momentum. Therefore, the penetration portion 80 can surely unseal the bottle 76 with small force.

### Seventh Embodiment

Next, a seventh embodiment of the present invention will be described. Only differences from the first embodiment will be described below. The same constituent elements as in the first embodiment are designated with the same reference signs, and the description will be appropriately omitted.

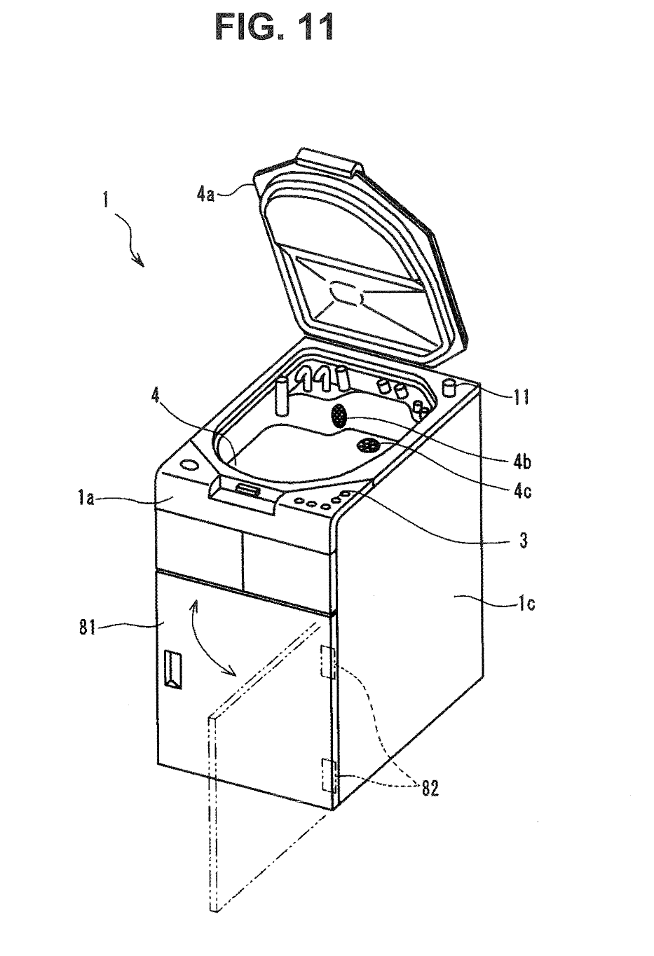

As shown in FIG. 11, the endoscope reprocessor 1 of the present embodiment includes, on a front surface, a front surface door 81 that opens forward. The front surface door 81 is a so-called single-swing door, and in an example of the present embodiment, the front surface door 81 rotates around a vertical axis based on hinges 82 provided on a right side facing the front surface of the apparatus main body 1a.

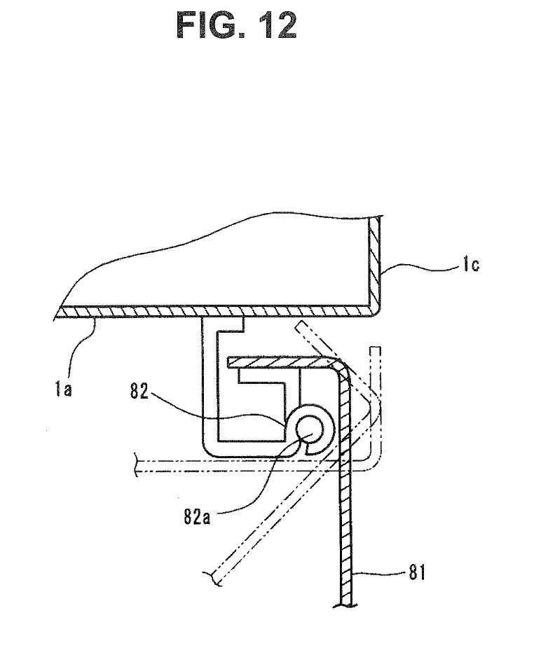

FIG. 12 is an enlarged view of the hinge 82 as viewed from above, parallel to a rotation shaft portion 82a. In FIG. 12, solid lines illustrate an open state of the front surface door 81 rotated 90° forward from the state in which the front surface door 81 is closed.

As shown in FIG. 12, the hinge 82 of the present embodiment is configured such that the front surface door 81 does not protrude outside (right side in the present embodiment) of a side surface 1c of the main body portion 1a when the front surface door 81 is rotated between the closed state and the open state.

Therefore, the front surface door 81 does not interfere with an apparatus, a wall, and the like near the side surface 1c in the present embodiment. That is, the endoscope reprocessor 1 of the present embodiment can be arranged in a space with a narrower breadth.

### Eighth Embodiment

Next, an eighth embodiment of the present invention will be described. Only differences from the first embodiment will be described below. The same constituent elements as in the first embodiment are designated with the same reference signs, and the description will be appropriately omitted.

The endoscope reprocessor 1 of the present embodiment is configured to conduct a water leak test of the endoscope 100. The water leak test is a test for checking that there is no puncture on outer skins and conduit walls for keeping the inside of the endoscope 100 airtight.

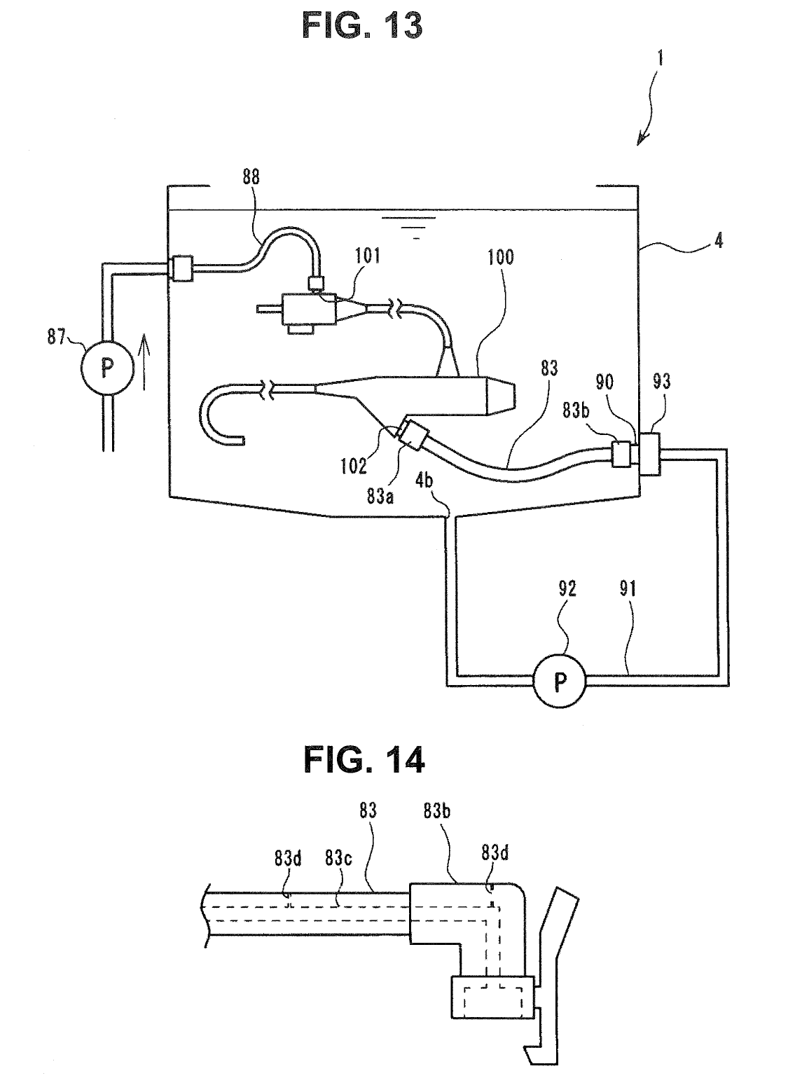

As shown in FIG. 13, the endoscope reprocessor 1 includes a pressure pump 87 and a pressure tube 88 that are components for sending a gas into the internal space of the endoscope 100 from a test pipe sleeve 101 provided on the endoscope 100. The pressure tube 88 is a conduit that can be connected to the test pipe sleeve 101, and the pressure pump 87 sends the air into the internal space of the endoscope 100 through the pressure tube 88.

In the water leak test of the present embodiment, water is accumulated in the processing tank 4 to submerge the endoscope 100, and then the pressure pump 87 is operated to send the air into the internal space of the endoscope 100 as shown in FIG. 13. At this point, a distal end portion 83a of a connection tube 83 is connected to a pipe sleeve 102 of the conduit included in the endoscope 100. A proximal end portion 83b of the connection tube 83 is connected to a connector portion 90 installed in the processing tank 4.

The connector portion 90 is connected to the circulation port 4b through a circulation conduit 91. A circulation pump 92 provided on the circulation conduit 91 operates to discharge the liquid in the processing tank 4 from the connector portion 90. A valve 93 for opening and closing the circulation conduit 91 is installed near the connector portion 90. The valve 93 is in a closed state at the execution of the water leak test.

As shown in FIG. 14, the connection tube 83 is provided with a channel 83c penetrating inside from the distal end portion 83a to the proximal end portion 83b. The connection tube 83 is connected to the pipe sleeve 102 and the connector portion 90, and the conduit of the endoscope 100 and the circulation conduit 91 are communicated through the channel 83c. Here, minute through holes 83d from an outer surface to the channel 83c are formed on the connection tube 83. One or a plurality of through holes 83d may be provided on the connection tube 83. The through holes 83d are provided at positions sunk in the water stored in the processing tank 4 at the execution of the water leak test.

When there is a puncture on the conduit wall of the endoscope 100, the air leaks into the conduit from the pressured internal space of the endoscope 100 if the water leak test is carried out. In the present embodiment, the distal end portion 83a of the connection tube 83 is connected to the pipe sleeve 102 of the conduit at the execution of the water leak test, and the side of the proximal end portion 83b of the connection tube 83 is closed by the valve 93. Therefore, the air leaked into the conduit from the puncture of the conduit wall moves to the connection tube 83 in the water leak test, and the air leaks from the through holes 83d of the connection tube 83. The through holes 83d are provided at positions sunk in the water, and the user can visually check bubbles generated by the leak of the air from the through holes 83d.

In this way, in the present embodiment, whether there is a puncture on the conduit wall of the endoscope 100 can be determined based on whether bubbles are generated at the parts provided with the through holes 83d of the connection tube 83 at the execution of the water leak test.

Note that the present invention is not limited to the embodiments, and the present invention can be appropriately changed within the scope and the spirit of the inventions that can be read from the claims and the entire specification. The changed endoscope reprocessors are also included in the technical scope of the present invention.

What is claimed is:

1. An endoscope reprocessor comprising:
a filtering tool comprising:
a lead-in portion that leads in a fluid;
a lead-out portion that leads out the fluid;
a main body portion provided with a channel connecting the lead-in portion and the lead-out portion;
a filter portion that is provided in the channel and filters the fluid flowing through the channel; and
a first check valve that is provided between the filter portion and the lead-out portion in the channel and allows the fluid to pass only in a direction from the filter portion toward the lead-out portion;
a processing tank that houses at least one of an endoscope and an endoscope accessory;
a tank that stores a cleaning liquid or a disinfecting liquid;
a lead-out conduit that is detachably connected to the lead-out portion of the filtering tool and guides the fluid led out from the lead-out portion to the processing tank;
a second check valve with a distal end connected to the lead-out conduit, the second check valve allowing the fluid to pass only in a direction from a proximal end toward the distal end;
a first conduit with a distal end connected to the second check valve and a proximal end connected to the tank; and
an injection pump that transfers the cleaning liquid or the disinfecting liquid in the tank from the proximal end toward the distal end of the first conduit.

2. The endoscope reprocessor according to claim 1, further comprising
a third check valve that is provided in a section of the lead-out conduit from a distal end to a part connected with the second check valve and that allows the fluid to pass only in a direction from the lead-out portion toward the processing tank in the lead-out conduit.
